(19) United States
(12) Patent Application Publication
(10) Pub. No.: US 2013/0260397 A1
(43) Pub. Date: Oct. 3, 2013

(54) APPARATUS FOR SAMPLING OF A REPRESENTATIVE AND NON-DESTRUCTIVE SAMPLE OF PARTICLES FROM BULK MATERIAL AS WELL AS A METHOD FOR SAMPLING USING THE APPARATUS

(71) Applicants: Scarlett Biselli, Hamburg (DE); Ngnintendem James Nkengfack, Hamburg (DE)

(72) Inventors: Scarlett Biselli, Hamburg (DE); Ngnintendem James Nkengfack, Hamburg (DE)

(73) Assignee: EUROFINS WEJ CONTAMINANTS GMBH, Hamburg (DE)

(21) Appl. No.: 13/821,199

(22) PCT Filed: Sep. 7, 2011
(86) PCT No.: PCT/DE11/75213

§ 371 Date: Jun. 18, 2013

(30) Foreign Application Priority Data
Sep. 9, 2010 (DE) 10 2010 037 425.3

(51) Int. Cl. G01N 33/04; G01N 33/02

(52) U.S. Cl. CPC G01N 33/04; G01N 33/02
USPC 435/7.92; 435/287.2; 73/61.71; 73/61.52; 73/61.72

(57) ABSTRACT

The present invention relates a method for the detection of at least one mycotoxin contamination from bulk ware, an apparatus for taking a sample of particles from bulk ware as well as a method for taking particles from bulk ware using the apparatus according to the invention and the use of the apparatus according to the invention.

This application is a national phase application under 35 U.S.C. §371 of International Application No. PCT/DE2011/075213, filed Sep. 7, 2011, which claims priority to German Application No. DE 10 2010 037 425.3, filed Sep. 9, 2010. The entire text of each of the above referenced disclosures is specifically incorporated herein by reference.

The present invention concerns a method for detecting at least one mycotoxin contamination in bulk material, an apparatus for sampling of a sample of particles from the bulk material as well as a method for sampling of particles from bulk material using the apparatus according to the invention and use of the apparatus according to the invention.

Mycotoxins are secondary metabolic products from moulds. Mycotoxins exert toxic effects already in smallest amounts. Thus, mycotoxins in food and feed represent a serious health risk. The effects of mycotoxins in mammals may be very diverse. It is known that they may have a carcinogenic, neurotoxic or immune suppressive effect in mammals. Besides of this, allergic reactions against mycotoxins have been described as well. Currently numerous mycotoxins are known which may be produced by various fungus species. Known mycotoxins are among others aflatoxin, ergot alkaloids, ochratoxin A (OTA), fusarium toxins such as deoxynivalenol (DON) and zearalenone (ZEA) and penicillium toxins such as citrinin. In this connection, ZEA and DON represent for example the main mycotoxins in grain, OTA is the main mycotoxin in green coffee, whereas nuts are mainly contaminated with aflatoxins. In view of the serious health risk, which is exerted by mycotoxins, extensive efforts have been made during the last years to develop and amend analysis methods in this field. In general, liquid chromatographic methods including mass spectrometric and fluorescence detection respectively are used as well as immuno assays (Schuhmacher et al., 2008; Shephard, 2009; Maragos and Busman, 2010). Moreover, food may be polluted with other contaminations or impurities. Respective examples are toxic metals or metal compositions such as arsenic, antimony or zinc, plant protection products, such as pesticides, fungicides, insecticide or herbicides or genetically altered organisms.

In view of the fact that mycotoxins as well as other contaminations or harmful residuals are distributed very heterogeneously in food and feed, especially the sampling and preparation of a sample of food, which has to be analyzed, represents a great challenge (Biselli et al., Mycotoxin Res. 24 (2), (2008), 98-104; Miraglia et al., Food Addit. Contam. Suppl 1, (2005), 31-36). According to the current approach prescribed by foodstuff regulation according to Regulation EC 401/2006 “Methods of sampling and analysis for the official control of the levels of mycotoxins in foodstuffs”, the homogenisation of several kilogram of loose bulk material, which is so-called bulk ware, is foreseen. Since several matrix components deteriorate the analysis procedure and may in particular complicate or even avoid a quantification of the mycotoxins, usually relatively expensive and complex purifications processes are conducted after the homogenisation, such as solid phase extraction, solid phase microextraction or an affinity chromatography (Senyuva and Gilbert, J. Chromatogr. B Analyt Technol Biomed Life Sci, 878 (2), (2010), 115-32; Biselli and Hummert, Food Addit. Contam. 22 (8), (2005), 752-60). In summary, sampling and sample preparation are thus characterized by a greater time and effort which is required.

Different sampling lances have been already described for an easier sampling. In EP 0 411 932 B1, a sampling probe for the sampling of a representative partial sample of grain and flour is described. In EP 1 221 605 B1 a portable spare-shaped sampling lance for sampling of a dust sample is described.

U.S. Pat. No. 6,324,927 B1 describes a method and apparatus for the sampling of contaminants, wherein the sample is exposed to vibrations within an airtight chamber in the presence of a carrier gas, which results in the release of dust particles.

Stroka et al., Toxicology Letters, 153 (2004), 99-107 describe the sampling of different food bulk ware by using a sample lance and the DiscoveryCERT FQS™ sampling systems respectively. The amount of dust of the sample is collected on different filter materials and analyzed using high performance liquid chromatographic (HPLC) as well as Fourier-transformations-IR-spectroscopy (FTIR) methods. However, a clear and unambiguous correlation of the contaminations of the dust and the bulk sample can not be determined.

Disadvantageous at the presently known methods is that these methods require the sampling using glass fibre filter or membranes respectively. However, this kind of sampling allows only an analysis of the sample in form of one single analysis. A repetitive verification or repetition of the analysis of the collected sample is thus not possible. Furthermore, a clear correlation in respect to the contamination with mycotoxins or other contaminations of the taken sample in comparison to the overall sample can not be determined using the taken sample according to the prior art. Accordingly, the obtained samples resulting from the presently available methods do not constitute representative samples. Therefore, the significance of such a sample is clearly lowered, since the sample does not provide an unambiguous hint concerning the real contamination with mycotoxins or other contaminations. Furthermore, it is also disadvantageous of the presently known methods, that these methods are in part technically very complex. This involves potential sources of errors and causes additionally high costs for the acquisition and operation of such apparatuses.

In view of the above-mentioned drawbacks of the methods of the prior art, the technical problem underlying the present invention is to enable an amended and representative sampling for food and feed bulk material, so-called bulk ware. Furthermore, the technical problem underlying the present invention is the provision of a method which can be easily conducted, which method is inexpensive and less labour-intensive. Moreover, the technical problem underlying the present invention is the provision of a reliable and representative method for the detection of a mycotoxin contamination.

This underlying technical problem is solved according to the subject-matter as defined in the claims.

A first subject-matter of the present invention relates to a method for the detection of at least one mycotoxin contamination in bulk material, the method including the steps of: (a) taking a sample of particles with a particle size of 0.1 mm and 1.0 mm from bulk material; (b) analysing of the taken particles; and (c) detecting of a mycotoxin contamination by determining the concentration of the mycotoxin contamination.

According to the invention, “contaminations” are understood in particular as impurities of food. Such impurities include mycotoxins, toxic metals or metal compositions, such as arsenic, antimony or zinc, plant protection products, such as pesticides, fungicides, insecticide or herbicides or genetically altered organisms.

Mycotoxins include in particular ochratoxins, aflatoxins, ergot alkaloids, fusarium toxins, such as particularly deoxynivalenol (DON) and zearalenone (ZEA), alternaria toxins and citrinin.

According to the invention the mycotoxins in the method of the present invention for the detection of at least one mycotoxin concentration is preferably selected from the group consisting of ochratoxins, aflatoxins, ergot alkaloids, fusarium toxins, such as in particular deoxynivalenol (DON) and zearalenone (ZEA), alternaria toxins and citrinin.

In a preferred embodiment of the invention it is foreseen that the particle size of the taken sample has a size of 0.1 mm to 0.7 mm, further preferred of 0.2 mm to 0.5 mm.

In a further preferred embodiment the analysis is conducted by high performance liquid chromatography (HPLC), ultra-high performance chromatography (UPLC), Fourier-transformations-IR-spectroscopy (FTIR), nuclear magnetic resonance (NMR), Direct Analysis in Real Time (DART) or Enzyme-linked Immunosorbent Assay (ELISA).

A further subject-matter of the present invention is an apparatus for sample taking of a sample of particles from bulk material with a separation unit (100) and a suction unit (110) to take the sample by an air stream leading from the separation unit to the suction unit, including a channel (20) with an upstream directed opening (25), in which a sonde (30) is arranged with a sieve (35), a cyclone (40) arranged downstream from the channel (20) and vertically to the channel (20) with a container (45) disposed downwards of the cyclone (40), a channel (50) conducted into the cyclone (40), the channel (50) is directed downstream to a filter (55), a discharging channel (60) downstream of the filter (55), which channel (60) conducts from the filter (55) to the suction unit (110), with a valve (65), and a channel (70) branching of from the channel (60), with a manometer (75) arranged at the end of the channel (70), wherein the suction unit (110) is an air blower, which exerts a velocity of the air stream in the channel (20) of preferably 30 to 70 m/s.

In the context of the present invention, “bulk material” is understood as a mixture of grainier or piece form of loose single goods, wherein the bulk material is in free-flowing form. According to the invention, “bulk material” is particularly understood as food and feed bulk material, such as grains of cereals, for example wheat, corn, rye, or barley, as well as flour, salt, sugar, nuts, or coffee.

According to the present invention, “upstream” is understood as an orientation which has a larger distance in view of the length and the dimensional relation of the suction unit and the therein produced air stream. According to the invention, “downstream” is understood as an orientation, which has a lower distance in view of the length and the dimensional relation of the suction unit and the therein produced air stream.

According to the invention, “vertical” is understood as a direction, which has essentially an angle of 90°. Furthermore according to the invention “vertical” is also understood as a direction, which has an angle deviating from 90°, for example in a range of 45° to 135°. According to the invention it is foreseen, that a vertical direction is understood as such a direction, in which the channel (20) is disposed such in relation to the cyclone (40), that the air stream including the particles reaches the cyclone (40) in such a way, that the particle are introduced tangentially and thus get on a circular track in the cyclone.

In connection with the present invention, it is foreseen that “channel” is understood as a connection, in particular a tube-like connection such as a tube, hose, or a pipe.

Contaminations, such as contaminations with mycotoxins, are generally very inhomogeneously distributed within the bulk material. This means that contaminations, in particular mycotoxins, are distributed inconsistently and irregularly within the bulk material. Therefore, one can not assume that already by taking of an arbitrary sample a contamination is correctly detected, since there is a high likelihood that said sample is indeed only contaminated to a lesser amount. Yet, it is possible that this does not reflect the real situation, since the overall bulk material is probably indeed contaminated in a definitely higher amount compared to what has been detected in a single sample. Accordingly in view of the inhomogeneous distribution of the contaminations, one has to fear that single samples each reflect different concentrations of contaminations. However, one has to conclude that a single sample is not significant and representative per se to deduce on this basis the real contamination of the overall bulk material.

In view if this, there are prejudices given concerning a method which relies on the taking of a single smaller sample, such as a particle sample, to determine the contamination of the overall bulk material in this way.

Surprisingly, it is however possible with the method according to the invention for the detection of at least one contamination in bulk ware to allow a reliable and significant detection of a contamination by the sample taking of a particle sample with a size of 0.1 mm to 1.0 mm. It could be shown that the particle sample has a contamination which indeed distinguishes from the contamination of the overall sample. Thus, the concentration of the contamination of the particle sample may be for example clearly higher compared to the concentration of contamination of the overall sample. Astonishingly, it could be shown that the taken particle sample with a particle size of 0.1 mm to 1.0 mm constitutes a representative particle sample, which provides information about the real concentration of the contamination of the overall sample. By correlating of the concentration of the contamination of the particle sample with the concentration of the contamination of the overall sample, it could be confirmed that the particle sample allows drawing an inference on the real contamination.

According to the invention, “reliable detection” or “significant detection” respectively is understood as a detection, which determines the presence of a food contamination in bulk material in a significant amount, such that a secured result according to statistical background is achieved.

Surprisingly, it could be shown that with the apparatus according to the invention samples of a distinct particle size can be taken, which particles, after an analysis in regard to mycotoxins as contaminants correlate in a distinct mycotoxin concentration in a high extent with the mycotoxin concentration of the overall sample of the bulk ware which has been analysed. This advantageous representative sampling is enabled by obtaining of the sample via the centrifugal separator, also called cyclone. In the context of the present invention, “representative” is understood as the characteristic of the sample to represent distinct features of a sample, such as contaminations with mycotoxins or plant or microbial toxins, in such a way, that said sample correlates with the overall sample to a high extent concerning these distinct features. According to the invention, the cyclone enables a separation of particles which are included in the air stream generated by the suction unit. In a preferred embodiment of the invention it is foreseen that the cyclone has a conical shape, which is tapered downwards. According to the invention it is further preferred foreseen that the air stream/particle mixture is introduced tangentially into the cyclone. Thereby the particles included within the air stream are directed on a circular track. Due to the tapering of the conical form of the cyclone, the speed of rotation of the air stream is elevated such that the particles are spinned against the conical wall of the cyclone due to the developing centrifugal force. Therefore, it is foreseen according to the present invention, that such particle samples which possess a distinct particle size are taken from the bulk ware which has to be analysed.

In a preferred embodiment of the invention, the adjustment of the air stream with a distinct suction force is enabled by the cyclone, whereby specifically particles with a desired size can be isolated and taken. It could be shown that particles with the distinct particle size can be isolated with the apparatus according to the invention, which particles have a high mycotoxin load. Mycotoxins are present on the surface in a notable amount. Particles having a lower diameter possess a higher surface/volume ratio in comparison to particles with a higher diameter. Accordingly particles having a lower size with a higher volume/surface ratio are loaded with mycotoxins in a higher extent compared to particles with a higher size and comparably lower volume/surface ratio. By the apparatus according to the invention, it is thus possible to take in particular samples of a particle size range, which represent in view of their mycotoxin concentration the overall load with mycotoxins in the bulk ware. Therefore, it is advantageously achieved with the apparatus according to the invention that very representative samples can be isolated by taking samples of a distinct particle size, whereby the load with mycotoxins in the overall bulk ware can be determined in an easy and reliable way.

According to the invention an apparatus is foreseen, wherein the separation unit (100) includes essentially a cyclone (40), a container (45), a channel (50), a filter (55), a channel (50), a filter (55), a valve (65), a channel (70), and a manometer (75).

In a preferred embodiment of the invention it is foreseen that a sonde (30) is introduced in a mobilisation unit (130) with an opening (135), from which the sample of the bulk ware is taken.

In a further preferred embodiment of the invention it is foreseen that the mobilisation unit (130) is a drum mixer, which has preferably a rotation speed of 10 to 120 rpm, preferably of 15 to 100 rpm, further preferred of 15 to 80 rpm, further preferred of 15 to 50 rpm, further preferred of 15 to 30 rpm, in particular preferred of 28 rpm. According to the invention it is foreseen that such a rotation speed allows a non-destructive sampling of the sample from the bulk ware. According to the invention, “non-destructive” is understood as the characteristic of a sample to be present in an intact and complete condition.

In a preferred embodiment of the invention it is foreseen that the mobilisation unit (130) is a vibrator, a vibrating conveyer or an air stream generated by a blower.

Preferably, the mobilisation unit (130) consists of iron or steel with a motor. Further preferred are metals, metal alloys or also plastics, polymers.

According to the invention it is preferred that the mobilisation unit (130) has a diameter of 20 to 100 cm, further preferred of 30 to 60 cm, in particular preferred of 50 cm.

According to the invention it is preferably foreseen that the sonde (30) has a diameter of 2 cm to 25 cm, further preferred of 4 cm to 10 cm, in particular preferred of 7 cm. According to the invention it is foreseen that an enlargement of the particle distance can be achieved via an enlargement of the diameter of the sonde. Accordingly, the use of a sonde with a bigger diameter allows the maintenance of a greater distance to the particles which have to be collected. Thereby it is possible that the particle sample can be taken with a dimensional flexible distance, without the necessity, that the sonde has to be placed in direct neighbourhood to the bulk ware which has to be analysed. Preferably the sonde (30) consists of polyethylene (PE). Alternatively preferred according to the present invention are further materials such as plastic, polymers, glass or metal.

According to the invention it is preferably foreseen that the sieve (35) in the sonde (30) is a metal sieve. According to the present invention it is further preferred that the sieve (35) is composed of plastic or a membrane. In a further preferred embodiment the sieve (35) has a mesh size of 1 mm to 10 mm, further preferred of 2 mm to 5 mm, in particularly preferred of 3 mm.

In a further preferred embodiment of the invention it is foreseen that the particle size fraction of the collected particles have a size of 0.1 mm to 1.0 mm, further preferred of 0.1 mm to 0.7 mm, in particular preferred 0.2 mm to 0.5 mm. According to the invention, it is preferred that consequently the particles of the collected sample have a size of 0.1; 0.2; 0.3; 0.4; 0.5; 0.6; 0.7; 0.8; 0.9 or 1.0 mm.

In a further preferred embodiment of the invention it is foreseen that the separation unit (100) has an outer dimension of 10 cm to 100 cm, further preferred of von 20 cm to 60 cm, in particular preferred of 40 cm.

In a preferred embodiment of the invention, it is foreseen that the suction unit (110) is an air blower, which generates in the channel (20) a speed of the air stream of preferably 40 to 60 m/s, in particularly preferred of 58 m/s. Further preferred according to the invention it is foreseen that the suction unit (110) has an outer dimension of 10 cm to 100 cm, further preferred of 20 cm to 60 cm, in particular preferred of 40 cm.

According to the invention it is foreseen that the deposition of a distinct particle fraction in the cyclone is achieved by the adjustment of an air stream with a distinct suction force. According to the invention, this adjustment is achieved with a reduction valve. The control of the adjustment of the suction stream is conducted with the manometer. A respective air stream with a distinct suction force can be adjusted, by the determined pressure.

According to the invention the channel (20) is preferably of plastic. Further preferred materials according to the invention are polymers, glass, metals or metal alloys. In a preferred embodiment of the invention it is foreseen that the channel (20) has a diameter of 1 cm to 5 cm, further preferred of 1.5 cm to 3.5 cm, in particularly preferred of 2.5 cm. Further preferred according to the invention, the channel (20) has a length of 0.1 m to 5 m, further preferred of 0.1 m to 3 m, in particularly preferred of 0.1 m to 2 m. In a preferred embodiment of the invention, the channel (20) is constructed in such a way at its downstream directed end that it is enabled that the particle/air stream mixture is blown in tangentially on a circular track into the cyclone (40).

In a preferred embodiment of the invention, it is foreseen that the cyclone (40) is composed of polyethylene. Further preferred according to the invention, the cyclone (40) is composed of other plastics or polymers glass, metals or metal alloys. According to the invention preferred, the cyclone (40) has a diameter of 1 cm to 25 cm, further preferred of 5 cm to 10 cm, in particular preferred of 8 cm. In a further preferred embodiment, the cyclone (40) has a height of 1 cm to 50 cm, further preferred of 1 cm to 30 cm, particularly preferred of 20 cm.

According to the invention, it is foreseen that the container (45) is a vessel for the collection of samples, which means a sampling storage vessel. In a preferred embodiment of the invention it is foreseen that the container (45) is connected to the cyclone (40) via a screw thread. Further connections, such as via grinding at the cyclone or at the container, as well as a clamp are also preferred according to the invention. According to the invention it is preferably foreseen that the container (45) is composed of polyethylene (PE). Further preferred according to the invention, the container (45) is composed of plastic, polymers or glass. According to the invention it is foreseen that the container (45) has a volume of 5 ml to 500 ml, further preferred of 100 ml to 400 ml, in particular preferred of 200 ml.

In a further preferred embodiment of the invention it is foreseen that an extracting agent is laid in advance into the container (45). According to the invention preferred extracting agents are acetonitrile, methanol or water.

Further preferred according to the invention are mixtures of two or three different extracting agents. According to a further preferred embodiment it is foreseen that two or three different extracting agents are present in different ratios in the mixture.

In a preferred embodiment, an acetonitrile/water mixture is used as extracting agent. In a further preferred embodiment the mixture ratio of acetonitrile/water is 80/20 v/v, further preferred acetonitrile/water 60/40 v/v, further preferred acetonitrile/water 50/50 v/v, further preferred acetonitrile/water 40/60 v/v, further preferred acetonitrile/water 20/80 v/v, and further preferred pure water.

In a further preferred embodiment of the invention it is foreseen that the extracting agent is acetonitrile/methanol/water. According to the invention further preferred is foreseen that acetonitrile/methanol/water is in a mixture ratio of 40/40/20 v/v/v.

According to the invention, it is foreseen that the extracting agent is adjusted correspondingly to the toxin which has to be detected or the material which has to be analysed respectively.

In a further preferred embodiment of the invention, it is foreseen that an analysis apparatus is connected directly to the container (45).

Further preferred according to the invention, it is foreseen that the analysis apparatus is suitable for the conduction of chromatographic, spectroscopic, or immunological procedure.

According to the invention it is foreseen that the analysis apparatus is suitable for the conduction of high performance liquid chromatography (HPLC), ultra-high performance liquid chromatography (UPLC), Fourier-transformations-IR-spectroscopy (FTIR), nuclear magnetic resonance (NMR), direct analysis in real time (DART) or immunoassay, in particular enzyme-linked immunosorbent assay (ELISA).

In a further preferred embodiment, it is foreseen that the upstream end of the channel (50) is directed vertically into the cyclone (40) in form of an emerging tube. In a further preferred embodiment of the invention it is foreseen that the channel (50) is composed of plastic. Further preferred according to the present invention it is foreseen that the channel (50) has a diameter of 1 cm to 5 cm, further preferred of 2 cm to 4 cm, particularly preferred of 2.5 cm.

In a further preferred embodiment of the invention it is foreseen that the filter (55) is a glass fibre filter. According to the invention it is further preferred foreseen that the filter (55) lies on a metal frit in a filter holder. According to the invention it is foreseen that the filter (55) avoids a pollution of the suction unit (110). In a further preferred embodiment of the invention, it is foreseen that the filter holder has a diameter of 1 cm to 20 cm, further preferred of 1 cm to 15 cm, particularly preferred of 8 cm. Further preferred it is foreseen that the filter (55) has a height of 1 cm to 20 cm, further preferred of 1 cm to 15 cm, in particular preferred of 8 cm.

In a preferred embodiment of the invention it is foreseen that the channel (70) is composed of plastic. Further preferred it is foreseen that the channel (70) has a diameter of 1 cm to 5 cm, further preferred of 2 cm to 4 cm, particularly preferred of 2.5 cm.

In a preferred embodiment of the present invention it is foreseen that the channel (60) is a hose. In a particular preferred embodiment of the present invention the channel (60) is a rubber hose.

In a further preferred embodiment of the present invention it is foreseen that the valve (65) is a reducing valve. According to the invention it is foreseen that the air stream generated from the suction unit (110) is regulated via the valve (65).

In a preferred embodiment of the invention it is foreseen that the cyclone (40), the container (45), the channel (50), the filter (55), the channel (70), the manometer (75), the downstream directed end of channel (20) as well as the upstream directed end of channel (60) are composed within a chamber (10) which has an upwards directed opening (15) and a downwards directed opening (16), wherein the downstream directed edge of channel (20) is directed through the opening (15) and the upstream directed edge of channel (60) is directed through the opening (16). According to the invention, it is preferably foreseen that the chamber (10) allows transportation and some stability of the apparatus according to the invention.

A further subject-matter of the invention is a method for taking a sample of particles of bulk ware using the apparatus for analysis of a mycotoxin contamination according to the invention, the method including the steps of:
(a) contacting the sonde (30) with particles from the bulk ware,
(b) aspirating of particles by generating an air stream from the suction unit (110) upstream over the cyclone (40) to the sonde (30), and
(c) collecting and taking of the particles in the container (45) disposed downwards of the cyclone (40), wherein the particles size fraction of the collected sample has preferably a size of 0.1 mm to 1.0 mm.

In a further preferred embodiment the size of the sample is from 0.1 mm to 0.7 mm, and further preferred from 0.2 mm to 0.5 mm.

In a further preferred embodiment of the invention, it is foreseen that the particles in step (a) are twirled by moving of the bulk ware.

According to the invention, it is foreseen that with the apparatus according to the invention as well as with the method according to the invention no direct contact of the sonde takes place by introducing it into the bulk ware. Moreover, it is foreseen that contacting is only conducted with particles of the bulk ware. In so far, the possibility is given by the apparatus according to the invention as well as the method according to the invention to take particles already at the loading or discharging of the bulk ware. According to the invention, it is preferred that contacting of the sonde with the particles is achieved by twirling using a mobilisation unit, such as a drum mixer, a vibrator, a vibrating conveyor or an air stream generated by a blower.

A further subject-matter of the present invention relates to the use of the apparatus of the invention for taking a representative sample from bulk ware for the detection of at least one mycotoxin contamination according to the present invention, wherein the particle size fraction of the collected sample has preferably a size of 0.1 mm to 1.0 mm.

Further preferred, the particle size fraction of the collected sample is a size of 0.1 mm to 0.7 mm, further preferred of 0.2 mm to 0.5 mm.

In a further preferred embodiment, the use according to the present invention is applied for the detection of mycotoxins.

Mycotoxins are preferably elected from the group consisting of ochratoxins, aflatoxins, ergot alkaloids, fusarium toxins, such as particularly preferred deoxynivalenol and zearalenone, alternaria toxins and citrinin.

The invention is further described in the following examples and the respective figures.

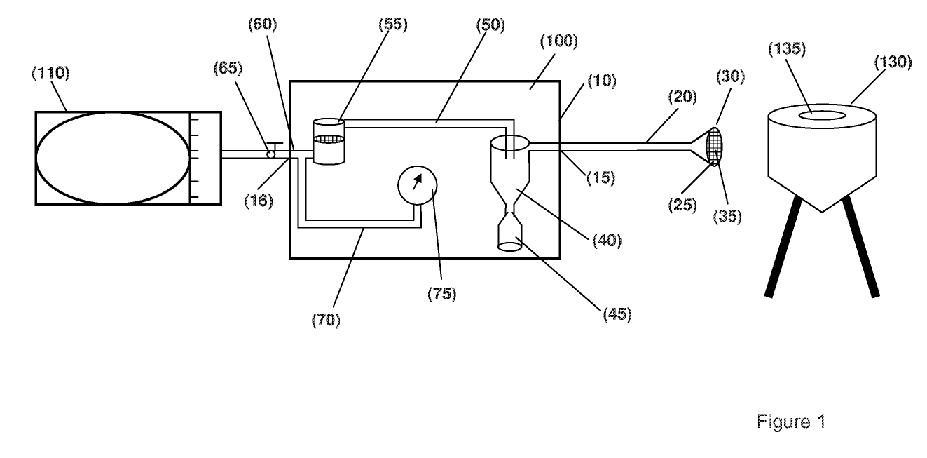

FIG. 1 shows a schematic drawing of a preferred embodiment of the apparatus according to the invention.

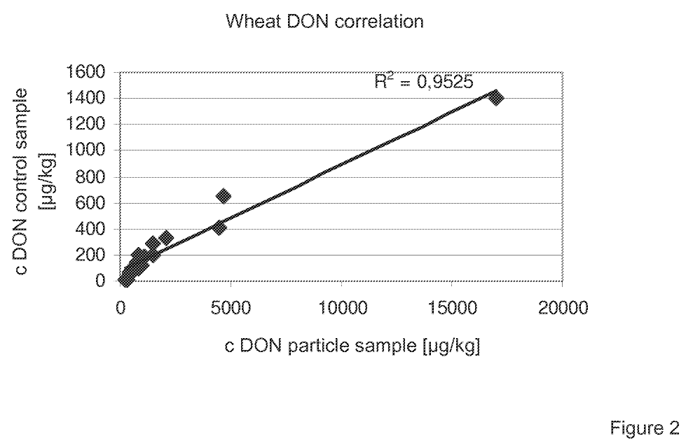

FIG. 2 shows the correlation of deoxynivalenol (DON) concentration [μg/kg] of particle samples, which have been obtained with the apparatus according to the invention, and control samples of wheat.

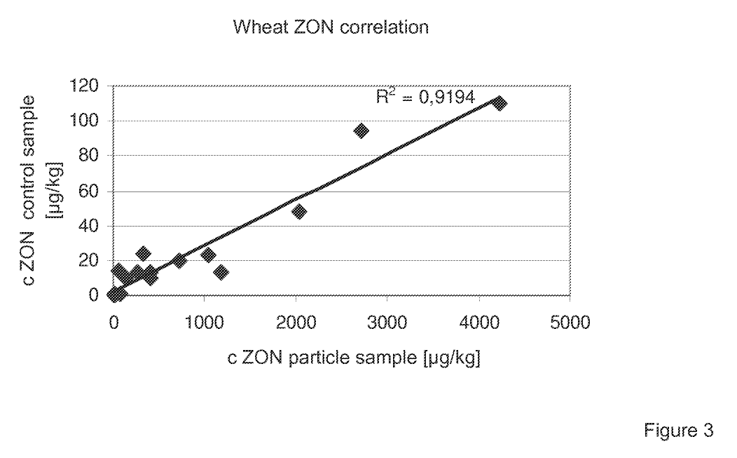

FIG. 3 shows the correlation of zearalenone (ZON) concentration [μg/kg] of particle samples, which have been obtained with the apparatus according to the invention, and control samples of wheat.

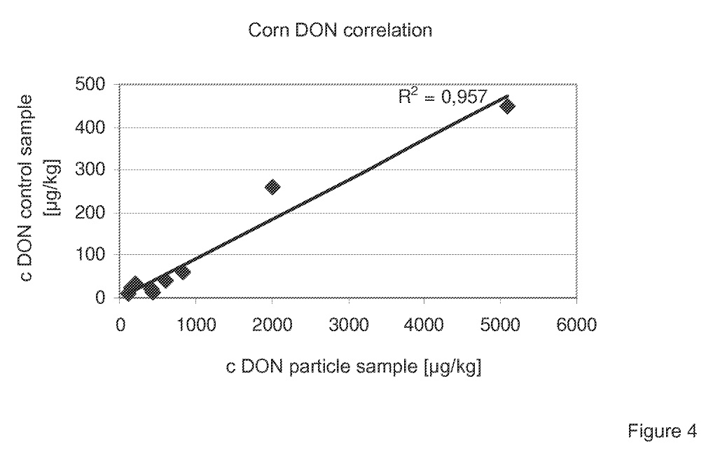

FIG. 4 shows the correlation of deoxynivalenol (DON) concentration [μg/kg] of particle samples, which have been obtained with the apparatus according to the invention, and control samples in corn.

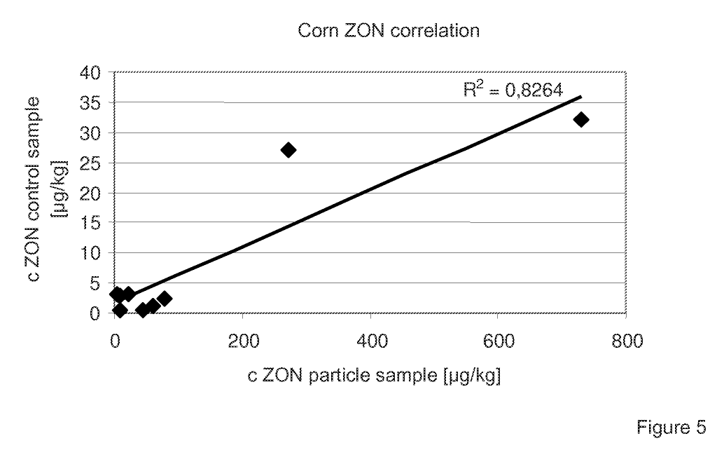

FIG. 5 shows the correlation of zearalenone (ZON) concentration [μg/kg] of particle samples, which have been obtained with the apparatus according to the invention, and control samples and corn.

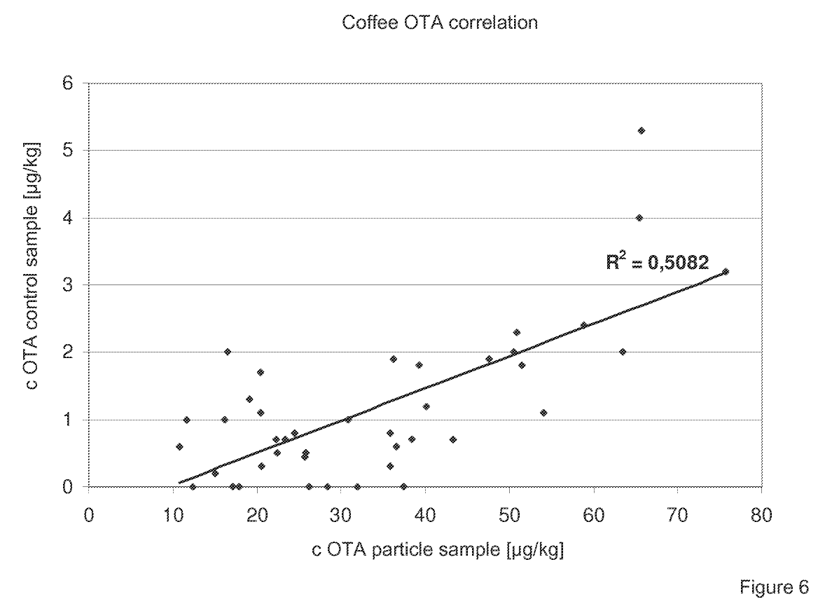

FIG. 6 shows the correlation of ochratoxin A (OTA) concentration [μg/kg] of particle samples, which have been obtained with the apparatus according to the invention, and control samples in coffee.

### EXAMPLE 1

### Taking of a Representative and Non-Destructive Sample of Particles from Bulk Ware Using an Apparatus According to the Invention

About 5 kg loose bulk ware (food bulk ware) of wheat, green coffee or nuts are introduced into a drum mixer. The drum mixer is used with a number of revolutions of 28 rpm. Thereby, particles are dissolved from the bulk ware and are twirled in the surrounding air. The sonde (30) of the apparatus according to the invention, which is shown in FIG. 1, is introduced into the mobilisation unit (130) within the opening (135). Starting from the air blower (110), an air stream is generated, which exerts a speed of 58.2 m/s in the directed tube (20), which is going downstream from the sonde (30). Thereby, the particles are sucked in and are directed from the sonde (30) through the tube (20), through the cyclone (40) to the sample collecting vessel (45), which is going downwards of the cyclone (40). With the distinct arrangement of the apparatus according to the invention, in particular of the cyclone (40), it is foreseen that in particular particles with a size of 0.2 mm to 0.5 mm are collected.

### EXAMPLE 2

### Analysis Method for Mycotoxins from Food Bulk Ware

### 2.1 Obtaining of the Particle Sample According to the Invention

From about 5 kg food bulk ware (wheat, malt, corn, green coffee, nuts, or similar bulk ware) particles are taken according to the method as described in example 1 using the apparatus according to the invention.

### 2.2 Sample Preparation of the Collected Particle Sample According to the Present Invention for Determining of the Mycotoxin Concentration

0.25-0.5 g of the particle sample are mixed with 10 ml of an acetonitrile/water mixture (80:20; v:v). As internal standards 200 μl of a zearalenon solution with the concentration of c=5 μg/ml as well as 200 μl of a fusarenon X solution with the concentration of c=20 μg/ml are endowed. The extraction is conducted over 5 minutes in ultra sonic bath at the highest level as well as 20 minutes on a mechanical shaker.

The extract is filtrated through a glass fiber filter. An aliquot of the extract is concentrated at 60° C. in a nitrogen stream and resolved in HPLC-eluents and diluted 1:5 with HPLC-eluents.

### 2.3 Homogenisation of the Control Samples for Conventional Analysis

2.3.1 Dry Ground Samples (e.g. Grain, Such as Wheat)

After the particle sample according to the invention has been obtained as described in 2.1, the remaining of the about 5 kg food bulk ware is removed from the drum mixer and directly finely ground (grain size<1 mm). These samples represent the controls for the particle samples as obtained with the apparatus according to the invention.

2.3.2 Wet Ground Samples (e.g. Green Coffee, Nuts, or Similar, However not Wheat, which is not Suitable for Wet Grinding Due to the Presence of Gluten, which would Otherwise Lead to the Development of Big Dough Lumps)

After the particle samples according to the invention are obtained as described in 2.1, the remaining of about 5 kg food bulk ware are removed from the drum mixer, and are homogenised according to the official analysis procedure of §64 LFBG L 00.00-111/1, mod. analysis of food—sample preparation procedure for the provision of an official sample, cross check and test for the determination of the mycotoxin amount in food—part 1: —Procedure for wet homogenisation. These samples represent the controls for the particle samples obtained using the apparatus according to the invention.

### 2.4 Sample Preparation of the Control Samples for Determining of the Mycotoxin Concentration

10 g of the control sample according to 2.3.1 and 2.3.2 respectively are mixed with 40 ml of a acetonitrile/water-mixture (80:20; v:v), and shaken for 30 minutes at 175 U/min. The extract is filtrated over a glass fiber filter. 1 ml of the filtrated extract is dissolved with 2 ml water, manually shaken and measured by HPLC-MS/MS.

### 2.5 Measurement of the Extracts of the Particle Samples According to the Invention and of the Control Samples

The measurement of the extracts is conducted by HPLC connected with the triple-quadropol-mass spectrometer. The stationary phase is a RP-18-phase. A binary gradient with eluent A and B is used. The flow rate is 0.8 ml/min.

### Gradient Program:

Eluent A: water/methanol (95+5; V+V), ammonium carbonate: 1 mmol/L, formic acid 0.0025%

Eluent B: methanol

| Time (min) | Eluent A % | Eluent B % |
|---|---|---|
| 0.00 | 80 | 20 |
| 6.9 | 20 | 80 |
| 7.0 | 20 | 80 |
| 8.0 | 0 | 100 |
| 10.0 | 0 | 100 |
| 10.1 | 80 | 20 |
| 14.0 | 80 | 20 |

The detection is conducted both in the positive (ochratoxin A, aflatoxin B1, T-2, HAT-2) and in the negative (deoxynivalenol, zearalenone) mode using the triple-quadropol-mass spectrometer as a special form of the HPLC-MS.

### EXAMPLE 3

### Analysis of the Mycotoxins Deoxynivalenol (DON) and Zearalenone (ZON) in Wheat Bulk Ware

Obtaining of the particle samples according to the invention is conducted according to example 1. The measurement and the determination of the concentration of DON and ZON respectively in the particle samples according to the invention as well as in the conventionally obtained control samples is conducted according to example 2. The results of the determination of the concentration of DON and ZON respectively as mycotoxins in the particle samples according to the present invention as well as in the control samples is shown in the following table 1:

TABLE 1

| | |
|---|---|
| deoxynivalenol (DON) | zearalenone (ZON) |

| | | | | |
|---|---|---|---|---|
| | Particle | Control | Particle | Control |
| Sample No. | sample | sample | sample | sample |

| | | |
|---|---|---|
| Wheat | μg/kg | μg/kg |

| | | | | |
|---|---|---|---|---|
| 673517 | 826.7 | 100 | 326.7 | 24 |
| 673518 | 1500 | 290 | 1040 | 23 |
| 673519 | 580 | 100 | 414 | 13 |
| 673521 | 2100 | 330 | 732 | 20 |
| 673522 | 1500 | 200 | 412 | 10 |
| 674444 | 950 | 120 | 144.3 | 10 |
| 676177 | 770 | 140 | 64.9 | 14 |
| 675915 | 460 | 62 | 17.4 | 0.3 |
| 676398 | 1100 | 190 | 1180 | 13 |
| 677547 | 4700 | 650 | 4220 | 110 |
| 678064 | 17000 | 1400 | 2710 | 94 |
| 682371 | 470 | 54 | 79 | 1 |
| 687390 | 260 | 13 | 19.8 | 1 |
| 687480 | 4500 | 410 | 2030 | 48 |
| 689724 | 310 | 15 | 16.9 | 0.1 |
| 718344 | 842 | 200 | 270 | 13 |

The results of the determination of the concentration are shown in FIGS. 2 and 3. Surprisingly, the inventors could show that the detection of mycotoxins in the particle samples obtained by the apparatus according to the invention correlate in a high amount with the results of the control samples obtained from the overall material. FIG. 2 shows a correlation of R²=0.9525 of the concentration of DON in the particle samples according to the invention and of the control samples. Since a perfect correlation would be assumed at a value of R²=1, the value of R²=0.9525 represents a very high correlation. In so far, the particle sample according to the invention represents a very representative sample, which reflects in a reliable amount the concentration of DON as mycotoxin in the overall sample. FIG. 3 shows that the correlation of the concentration of ZON in wheat of the particle samples according to the invention with the control samples is R²=0.9194. Thus, the obtained sample according to the present invention is also in a high amount significant in view of the measured concentration of the mycotoxin ZON in the control sample.

### EXAMPLE 4

### Analysis of the Mycotoxins Deoxynivalenol (DON) and Zearalenone (ZON) in Corn Bulk Ware

Obtaining of the particle samples according to the invention is conducted according to example 1. The measurement and the determination of the concentration of DON and ZON respectively in the particle samples according to the invention as well as in the conventionally obtained control samples is conducted according to example 2. The results of the measurement of the concentrations of DON and ZON respectively in the particle samples according to the invention and in the conventionally extracted control samples are shown in the following table 2:

TABLE 2

| Parameter | |
|---|---|
| DON | ZON |

| | | | | |
|---|---|---|---|---|
| Sample | Particle | Control | Particle | Control |
| No. | sample | sample | Sample | sample |

| | | |
|---|---|---|
| Corn | μg/kg | μg/kg |

| | | | | |
|---|---|---|---|---|
| 684007 | 110 | 10 | 9.73 | 0.4 |
| 688723 | 440 | 14 | 78.2 | 2.5 |
| 688726 | 410 | 21 | 44.4 | 0.4 |
| Mais_Lab | 2000 | 260 | 272 | 27 |
| 689723 | 5100 | 450 | 730 | 32 |
| 691361 | 840 | 61 | 60.9 | 1.2 |
| 692053 | 150 | 25 | 9.1 | 2.9 |
| 692054 | 210 | 35 | 5.4 | 3.1 |
| 692055 | 605 | 42 | 22.1 | 3.1 |

The inventors could surprisingly show that also in corn as food bulk ware a high correlation is given between the obtained particle samples according to the invention and the directly ground control samples from the corn bulk ware in regard to the concentrations of DON and ZON respectively. FIG. 4 shows a correlation of R²=0.957 for the concentration of DON. Thus, the sample obtained by the apparatus according to the invention is highly significant and thus representative, to determine in such a way the contamination of corn with the mycotoxin DON in a reliable and easy way. The same applies also to the measurement of the concentration of ZON. FIG. 5 shows a high correlation of R²=0.8264. Thus, it is also shown for the determination of this further mycotoxin ZON that a sample can be obtained by the apparatus according to the invention, which sample is reliable and representative in regard to its significance. In comparison to the time intensive and technically complex samples, which have to be obtained by the complete homogenisation of the bulk ware, the sample of particles from the bulk ware according to the invention thus represents an alternative, which is assured and technically easier to manage, and moreover results in time and cost saving and represents at the same time comparable results concerning the mycotoxin concentration.

### EXAMPLE 5

### Analysis of Mycotoxin Ochratoxin A (OTA) in Coffee Bulk Ware

Obtaining of the particle samples according to the invention is conducted according to example 1. The measurement and the determination of concentration of ochrotoxin A (OTA) in the particle samples according to the invention as well as in the conventionally obtained control samples is conducted according to example 2. The results of the measurement of the concentrations of ochratoxin A (OTA) in the particle samples according to the invention and in the conventionally obtained control samples are shown in the following table 3:

TABLE 3

| | Ochratoxin A | Ochratoxin A |
|---|---|---|
| | (OTA) | (OTA) |
| Sample | Particle | Control |
| No. | Sample | sample |
| Coffee | [μg/kg] | [μg/kg] |
| 683699 | 40.1 | 1.2 |
| 683700 | 50.5 | 2.0 |
| 684131 | 20.4 | 1.7 |
| 684132 | 25.7 | 0.5 |
| 684970 | 24.4 | 0.8 |
| 685794 | 22.3 | 0.7 |
| 685795 | 15.1 | 0.2 |
| 685796 | 10.8 | 0.6 |
| 686195 | 16.1 | 1.0 |
| kaffee_Lab | 26.1 | 0.0 |
| 686652 | 63.5 | 2.0 |
| 686653 | 20.6 | 0.3 |
| 687580 | 65.5 | 4.0 |
| 687951 | 11.6 | 1.0 |
| 687952 | 47.6 | 1.9 |
| 687953 | 39.3 | 1.8 |
| 687954 | 51.5 | 1.8 |
| 688832 | 65.7 | 5.3 |
| 689831 | 75.7 | 3.2 |
| 688425 | 20.4 | 1.1 |
| 690546 | 50.9 | 2.3 |
| 690547 | 37.4 | 0.0 |
| 690969 | 54.1 | 1.1 |
| 690970 | 36.3 | 1.9 |
| 694987 | 17.8 | 0.0 |
| 694988 | 32.0 | 0.0 |
| 694525 | 12.3 | 0.0 |
| 701399 | 23.37 | 0.70 |
| 706186 | 28.39 | 0.00 |
| 706187 | 58.88 | 2.40 |
| 706495 | 36.53 | 0.60 |
| 706496 | 43.26 | 0.70 |
| 707928 | 17.15 | 0.00 |
| 708735 | 16.56 | 2.00 |
| 709169 | 30.87 | 1.00 |
| 709710 | 19.10 | 1.30 |
| 710087 | 25.83 | 0.50 |
| 711828 | 35.83 | 0.80 |
| 712250 | 22.37 | 0.50 |
| 712249 | 35.82 | 0.30 |
| 712639 | 38.35 | 0.70 |

The measured values of table 3 show a correlation between the load with ochratoxin A (OTA) in the particle samples according to the invention with the control samples. This correlation is shown in FIG. 6. The correlation is R²=0.5082. This value can be considered as relatively low. A reason for this is a low overall contamination of the analysed coffee bulk ware. Nevertheless, it is possible to draw the conclusion that an OTA contamination of the particle samples according to the invention of <50 μg/kg means an overall contamination of the overall sample with OTA of <5 μg/kg.

1. A method for the detection of at least one mycotoxin contamination in bulk ware, the method including the steps of:
(a) taking a sample of particles with a particle size of 0.1 mm to 1.0 mm from bulk ware;
(b) analysing of the taken sample;
(c) detecting at least one mycotoxin contamination by determining of the concentration of the mycotoxin contamination, and
(d) inferring the actual mycotoxin contamination of the bulk ware by correlating the concentration in the sample as determined in step (c) with the concentration of the overall sample of the bulk ware.

2. The method according to claim 1, wherein the particle size of the extracted sample has a size of 0.1 mm to 0.7 mm, further preferred of 0.2 mm to 0.5 mm.

3. The method according to claim 1, wherein the analysis is conducted by High Performance Liquid Chromatography (HPLC), Ultra-High-Performance Chromatography (UPLC), Fourier-Transformations-IR-Spectroscopy (FTIR), Nuclear magnetic resonance (NMR), Direct Analysis in Real Time (DART) or enzyme-linked immunosorbent assay (ELISA).

4. The method according to claim 1, wherein the mycotoxins are selected from the group consisting of ochratoxins, Aflatoxins, ergot alkaloids, fusarium toxins, in particular preferred the deoxynivalenol and zearalenone, alternia toxins and citrinin.

5. An apparatus for taking a representative sample of particles from bulk ware for the detection of a mycotoxin contamination with a separation unit (100) and a suction unit (110), to take the sample by an air stream directed from the separation unit to the suction unit, including a channel (20) with an upstream directed opening (25), in which a sonde (30) is arranged with a sieve (35), a cyclone (40), arranged downstream from channel (20) and vertically to the channel (20) arranged, with a container (45) disposed downwards of the cyclone (40), a channel (50) conducted into the cyclone (40), the channel (50) is directed downstream to a filter (55), a discharging channel (60) downstream of the filter (55), which channel (60) conducts from the filter (55) to the suction unit (110), with a valve (65), and a channel (70), branching of from the channel (60), with a manometer (75) arranged at the end of the channel (70), wherein the suction unit (110) is an air blower, which exerts a velocity of the air stream in the channel (20) of preferably 30 to 70 m/s.

6. The apparatus according to claim 5, wherein the sonde (30) is directed into a mobilisation unit (130) with an opening (135), from which the sample from the bulk ware is extracted.

7. The apparatus according to claim 5, wherein the mobilisation unit (130) is a drum mixer.

8. The apparatus according to claim 5, wherein the mobilisation unit (130) is a vibrator, a vibrating conveyer, or an air stream exerted by a blower.

9. The apparatus according to claim 5, wherein the particle size fraction of the extracted sample has a size of 0.1 mm to 1.0 mm, 0.1 mm to 0.7 mm, or 0.2 mm to 0.5 mm.

10. The apparatus according to claim 5, wherein the speed of the air stream is from 40 to 60 m/s or of 58 m/s.

11. The apparatus according to claim 5, wherein an extracting agent is laid into the container (45).

12. The apparatus according to claim 5, wherein an analysis apparatus is directly connected to the container (45).

13. The apparatus according to claim 5, wherein the analysis apparatus is configured for the conduction of chromatographic, spectroscopic or immunological procedures.

14. The apparatus according to claim 13, wherein the analysis apparatus is configured for the conduction of High Performance Liquid Chromatography (HPLC), Ultra-High-Performance-Chromatography (UPLC), Fourier-Transformations-IR-Spectroscopy (FTIR), Nuclear Magnetic Resonance (NMR), Direct Analysis in Real Time (DART) or Enzyme-Linked Immunosorbent Assay (ELISA).

15. The apparatus according to claim 5, wherein the cyclone (40), the container (45), the channel (50), the filter (55), the channel (70), the manometer (75), the downstream directed end of channel (20) as well as the upstream directed end of channel (60) are arranged within a chamber (10), which has an upstream directed opening (15) and a downstream directed opening (16), wherein the downstream directed end of channel (20) is directed to the opening (15) and the upstream directed end of channel (60) is directed to the opening (16).

16. A method for taking a representative sample of particles from bulk ware using an apparatus according to anyone of the claims 5 for the detection of at least one mycotoxin contamination, the method including the steps of:
(a) contacting the sonde (30) with particles from the bulk ware,
(b) aspirating of the particles by generating of an air stream from the suction unit (110) upstream over the cyclone (40) to the sonde (30), and
(c) collecting and taking of the particles in the container (45) disposed downwards of the cyclone (40).

17. A method according to claim 16, wherein the particles in step (a) are twirled by moving of the bulk ware.

18. A method according to claim 16, wherein the particle size fraction of the collected sample has a size of 0.1 mm to 0.7 mm, particularly preferred 0.2 mm to 0.5 mm.

19. A method using an apparatus according to claim 5 comprising extracting a representative sample from bulk ware to detect at least one mycotoxin contamination.

20. The method according to claim 19, wherein the particle size fraction of the collected sample has a size of 0.1 mm to 1.0 mm, 0.1 mm to 0.7 mm, or 0.2 mm to 0.5 mm.

21. The method according to claim 19, wherein the mycotoxins are selected from the group consisting of ochratoxins, aflatoxins, ergot alkaloids, zearalenone, and trichothecenes.

22. The method of claim 7, wherein the rotation speed is 10 to 120 rpm, 15 to 100 rpm, 15 to 80 rpm, 15 to 50 rpm, 15 to 30 rpm, or 28 rpm.

23. The method of claim 16, wherein the particles size fraction of the collected sample has a size of 0.1 mm to 1.0 mm

24. The method according to claim 16, wherein the mycotoxins are selected from the group consisting of ochratoxins, aflatoxins, ergot alkaloids and fusarium toxins.

25. The method according to claim 24, wherein the mycotoxins are selected from the group consisting of deoxynivalenol and zearalenone, alternaria toxins and citrinin.

26. The method according to claim 21, wherein the mycotoxins are selected from the group consisting of deoxynivalenol, alternaria toxins and citrinin.
